(19) United States
(12) Patent Application Publication
(10) Pub. No.: US 2010/0113380 A1
(43) Pub. Date: May 6, 2010

(54) P2X7 ANTAGONISTS TO TREAT AFFECTIVE DISORDERS

(76) Inventors: Natalie A. Bratcher, Chicago, IL (US); Lynne E. Rueter, Round Lake Beach, IL (US); Michael W. Decker, Mundelein, IL (US); Richard R. Harris, Evanston, IL (US); Michael F. Jarvis, Vernon Hills, IL (US); William A. Carroll, Evanston, IL (US); Ana M. Basso, Libertyville, IL (US)

Correspondence Address: Robert DeBerardine;D-377/AP6A-1, Abbott Laboratories, 100 Abbott Park Road, Abbott Park, IL, 60064-6008, US

(21) Appl. No.: 12/581,525

(22) Filed: Oct. 19, 2009

Related U.S. Application Data
(63) Continuation of application No. 11/694,431, filed on Mar. 30, 2007, now abandoned.
(60) Provisional application No. 60/787,825, filed on Mar. 31, 2006.

(51) Int. Cl. A61K 31/70

(52) U.S. Cl. 514/47

(57) ABSTRACT

The present invention provides methods to treat mood disorders and anxiety disorders using antagonists of the P2X₇ receptor and pharmaceutical compositions thereof, or combinations.

This application claims priority to the provisional application Ser. No. 60/787,825 filed on Mar. 31, 2006.

### BACKGROUND OF THE INVENTION

P2X₇ receptors are ionotropic receptors activated by ATP, which may regulate neurotransmission in the CNS by activating presynaptic and/or postsynaptic P2X₇ receptors on central and peripheral neurons and glia (Deuchars S. A. et al., J. Neurosci. 21:7143-7152, (2001), Kanjhan R. et al., J. Comp. Neurol. 407:11-32 (1997), Le K. T. et al., Neuroscience 83:177-190 (1998)). Activation of the P2X₇ receptor on cells of the immune system (macrophages, mast cells and lymphocytes) leads to release of interleukin-1β (IL-1β), giant cell formation, degranulation, and L-selectin shedding. ATP is able to increase local release and process of IL-1 (□ and □) in rats through a P2X₇receptor mediated mechanism following lipopolysaccharide (LPS) intraperitoneal injections (Griffiths et al., J. Immunology Vol. 154, pages 2821-2828 (1995); Solle et al., J. Biol. Chemistry, Vol. 276, pages 125-132, (2001)). P2X₇ receptor antagonists as those described in International Patent Application WO05/111,003 as well as U.S. patent application Ser. Nos. 10/909,502 and 10/980,674 have been shown to decrease the secretion of IL-1β from THP-1 cultured cells.

It has been hypothesized that excessive secretion of macrophage monokines, IL-1, INF-□ and TNF-□, could be a potential cause for depression (Smith, R. S. Med Hypotheses Vol. 35, pages 298-306 (1991)).

Depression is one of several forms of mood disorders. Mood disorders are divided into Depressive Disorders (“unipolar depression”), Bipolar disorders, and two disorders based on etiology, i.e., Mood Disorder due to a general Medical Condition and Substance-Induced Mood Disorder (American Psychiatric Association: Diagnostic and Statistical Manual of Mental Disorders (DSM-IV-TR), Fourth Edition, Text Revision. Washington, D.C., American Psychiatric Association, pages 345-428, 2000). Comorbidity between mood disorders and anxiety disorders has a high rate of prevalence, and 50-60% of depressed patients report a lifetime history of anxiety disorders (Gorman, J. M. Depress. Anxiety Vol. 4, pages 160-8 (1996-97); Regier, D. A. et al., British Journal of Psychiatry Supplement Vol. 34, pages 24-8 (1998); Kaufman, J. and Charney D., Depress. Anxiety Vol. 12 Suppl. 1, pages 69-76 (2000)).

Patients with depression have higher levels of inflammatory cytokines than control patients (Mikova, O., et al. Eur Neuropsychopharmacol Vol. 11, pages 203-8 (2001); Tuglu, C., et al. Psychopharmacology (Berl) Vol. 170, pages 429-33 (2003); Penninx, B. W. et al. Biol Psychiatry Vol. 54, pages 566-72 (2003). In animals, central and systemic administration of IL-1 induces changes in behavior that are very similar to those described for depression (Connor, T. J. & Leonard, B. E. Life Sci Vol. 62, pages 583-606 (1998); Anisman, H. & Merali, Z. Adv Exp Med Biol Vol. 461, pages 199-233 (1999); Dantzer, R. Ann N Y Acad Sci Vol. 933, pages 222-34 (2001); Knosman et al Trends Neurosci Vol. 25, pages 154-9 (2002)). In addition, immunostimulation as well as administration of proinflammatory cytokines such as IL-1, activates the hypothalamus-pituitary-adrenal (HPA) axis (Sharp, B. M., et al., Endocrinology Vol. 124, pages 3131-33 (1989); Besedovsky, H. O. et al., Science Vol. 233, pages 652-4 (1986); Besedovsky, H. O. et al., J. Steroid Biochem. Mol. Biol., Vol. 40, pages 613-8 (1991)) and induces anxiety-like behaviors in experimental animals and humans (Lacosta S. et al. Brain Res. Vol. 818, pages 291-303 (1999); Connor, T. J. et al., Neuroscience Vol. 84, pages 923-33 (1998); Anisma, H. and Merali, Z., Adv. Exp. Med. Biol. Vol. 461, pages 199-233, (1999); Reichenberg, A. et al., Arch. Gen. Psychiatry Vol. 58, pages 445-52 (2001)).

Genetic linkage studies have revealed a strong association between P2X₇ receptors, depression, and bipolar disorders (Shink E. et al, Mol. Psychiatry 10:545-552 (2005), Shink E. et al., Am. J. Med. Genet. B Neuropsychiatr. Genet. 135:50-58 (2005)).

Some of the currently available antidepressants have anti-inflammatory properties and can reduce the release of pro-inflammatory cytokines, which may account (at least partially) for their therapeutic effect. In vitro, tricyclic antidepressants decrease spontaneous and LPS-induced secretion of IL-1, IL-6 and TNF- [symbol] (Xia, Z., et al. Immunopharmacology Vol. 34, pages 27-37 (1996). Repeated administration of the antidepressant imipramine, reduces IL-1 and IL-2 production and increases IL-10 production (a negative immunoregulatory cytokine) in the chronic mild stress model of depression (Kubera, M. et al. J Clin Psychopharmacol Vol. 21, pages 199-206 (2001); Kubera, M. et al. J Affect Disord Vol. 63, pages 171-8 (2001)).

Depression is the most common mental disease and the fourth most important cause of disability worldwide. It is expected that rates of depression in the population will be increased in the upcoming years. Despite multiple available treatments for depression, there are still several serious unmet needs. These include a need for improved efficacy, for better tolerability, for a more rapid onset of action and for prevention of relapse and recurrence of depressive episodes. Current antidepressant drugs are mainly based on the monoamine hypothesis of depression. Serotonin re-uptake inhibitors represent the first line of treatment, however although these compounds are safer and with less side effect than other antidepressants, no improvement in terms of efficacy, onset of action or prevention of relapse has been observed. The development of novel agents for the treatment of depression based on novel targets is needed.

Considering that cytokines like IL-1 can induce behavioral and physiological changes that resemble depression, that altered levels of pro-inflammatory cytokines are associated with the course of depression as well as the response to antidepressant treatments, and that P2X₇ receptors and P2X₇ receptor antagonists play an important role modulating the release of IL-1, it could be hypothesized that the blockade of P2X₇ receptors might result in antidepressant-like properties.

In view of the facts described above, the present application describes a novel target to treat mood disorders and anxiety disorders, namely the P2X₇ receptor, and antagonist of the P2X₇ receptor as novel drugs useful as therapeutic agents to treat mood and anxiety disorders.

### BRIEF DESCRIPTION OF THE FIGURES

FIG. 1. Comparison of wild type (WT) and P2X₇ receptor KO mice in the mouse Tail Suspension Test.

FIG. 2. Comparison of wild type (WT) and P2X₇ receptor KO mice in the mouse Forced Swim Test.

### DETAILED DESCRIPTION OF THE INVENTION

The present invention provides an alternative method to treat mood and anxiety disorders. Accordingly, the present invention relates to a method of using a modulator of the P2X₇ receptor, preferably an antagonist, to treat mood disorders, preferably depression, and anxiety disorders.

Specifically, the present invention encompasses a method of treating a patient suffering from a mood disorder, including those suffering from a treatment resistant form of depression, comprising administering a therapeutically effective amount of a modulator of P2X₇ receptor activity to a subject suffering from said affective disorder. It is understood that the mood disorder may be one among many of the disorders affecting mood and behavior. For example, mood disorders comprise depressive disorder (that includes major depressive disorder, dysthymic disorder), bipolar disorder (includes bipolar I disorder, bipolar II disorder, cyclothymic disorder), mood disorder due to a general medical condition and substance-induced mood disorder (American Psychiatric Association: Diagnostic and Statistical Manual of Mental Disorders (DSM-IV-TR), Fourth Edition, Text Revision. Washington, D.C., American Psychiatric Association, pages 345-428, 2000.). Preferably, the disorder is a depressive disorder. The present invention also encompasses a method of treating a patient suffering from an anxiety disorder. Anxiety disorders include: panic attack, agoraphobia, specific phobia, social phobia, obsessive-compulsive disorder, posttraumatic stress disorder, acute stress disorder, and generalized anxiety disorder.

This method comprises the use of a modulator of P2X₇ receptor activity. For the purpose of the present invention, a modulator is a compound or a combination of compounds that can modify the activity of the P2X₇ receptor after it interacts with or binds to the receptor. A modulator comprises agonists and antagonists. An agonist is a compound that can interact with the receptor and initiate a physiological or pharmacological response. The term agonist includes, full agonists, partial agonists and inverse agonists. An antagonist is a compound that interacts or binds with the receptor and reduces, blocks or inhibits the physiological or pharmacological response characteristic of the receptor. The present invention preferably refers to antagonists of the P2X₇ receptor.

Antagonists of the P2X₇ receptor can be identified by several methods known by those skilled in the art. For example, the inhibitory activity of the antagonists of the P2X₇ receptor can be determined by their capacity to inhibit the agonist-induced pore formation using the fluorescent dye YO-PRO and Fluorescence imaging Plate Reader (FLIPR) in THP-1 cells. In general, the agonist used in these identification procedures is BzATP. Similarly, for antagonist activity measurements, the percent maximal intensity induced by a specific concentration of BzATP, and the percent intensities induced in the present of increasing concentrations of antagonists are plotted against each concentration of compound to calculate IC₅₀ values. The potency of the antagonists is inversely proportional to their IC₅₀ value. Inhibitory biological activity of the antagonists can be determined by their effect in inhibiting agonist-induced IL-1 [symbol] release. In general, the agonist used in these identification procedures is BzATP. Increasing concentrations of antagonists are added before the agonists and the concentration of the antagonists that inhibited 50% of the agonist-released IL-1□ is expressed as IC₅₀. To understand the involvement of P2X₇ receptor and cytokines in depression, P2X₇ knockout mice can be used in animal models of depression and anxiety. The Forced Swim Test (FST) and Tail Suspension Test (TST) are considered “behavioral despair” models, based on the rationale that exposure to inescapable stress leads to hopelessness and despair (immobility). Typically mice will attempt to escape during the first few minutes and then show an immobile posture. Immobility is hypothesized to reflect a state of depressed or lowered mood and is reduced by a wide variety of antidepressants drugs.

The present invention also provides pharmaceutical compositions that comprise the modulator of P2X₇ receptor activity, preferably an antagonist of the present invention, a pharmaceutically acceptable salt or prodrug thereof, and one or more non-toxic pharmaceutically acceptable carrier or diluent.

The pharmaceutical compositions can be formulated for oral administration in solid or liquid form, for parenteral injection, for rectal or vaginal administration, and for topical, dermal or transdermal administration. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The term “parenteral,” as used herein, refers to modes of administration, including intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions.

Solid dosage forms for oral administration include capsules, tablets, pills, powders, and granules. Liquid dosage forms for oral administration include pharmaceutically acceptable emulsions, microemulsions, solutions, suspensions, syrups and elixirs. Dosage forms for topical or transdermal administration of a compound of this invention include ointments, pastes, creams, lotions, gels, powders, solutions, sprays, inhalants or patches. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate.

Also, the basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil-soluble or dispersible products are thereby obtained.

Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid, and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethyl ammonium, methylamine, dimethylamine, trimethylamine, triethylamine, diethylamine, ethylamine and the such as. Other representative organic amines useful for the formation of base addition salts include ethylenediamine, ethanolamine, diethanolamine, piperidine, and piperazine.

The term “pharmaceutically acceptable prodrug” or “prodrug,” as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of formula (I), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987). The invention contemplates pharmaceutically active compounds either chemically synthesized or formed by in vivo biotransformation to the compounds of the present invention.

The present invention also encompasses a method of treating mood and anxiety disorders comprising administering a therapeutically effective amount of the pharmaceutical composition of this invention, preferably to treat depression or anxiety, most preferably depression.

The present invention also encompasses a method for treating a patient suffering from, or susceptible to treatment resistant, mood disorders and anxiety disorders, comprising administering to said patient an effective amount of a first component which is a P2X₇ antagonist or a pharmaceutically acceptable salt thereof, in combination with an effective amount of a second component which is any known antidepressant, tranquilizer or neuroleptic agent, or a pharmaceutically acceptable salt thereof.

The present invention will be further clarified by the following examples, which are only intended to illustrate the present invention and are not intended to limit the scope of the present invention.

### EXAMPLE 1

### Inhibition of agonist-induced IL-1 Release

THP-1 cells were plated in 24-well plates at a density of 1×10⁶ cells/well/ml. On the day of the experiment, cells were differentiated with 25 ng/ml LPS and 10 ng/ml final concentration of □IFN for 3 hours at 37° C. In the presence of the differentiation media, the cells were incubated with the antagonists of the present invention for 30 minutes at 37° C. followed by a challenge with 1 mM BzATP for an additional 30 minutes at 37° C. Supernatants of the samples were collected after a 5 minutes centrifugation in microfuge tubes to pellet the cells and debris and to test for mature IL-1□ released into the supernatant using either R & D Systems Human IL-1□ ELISA assay or Endogen Human IL-1□ ELISA, following the manufacturer's instructions. The concentration of the antagonists that inhibited 50% of the agonist-release of IL- [symbol] is expressed as IC₅₀.

### EXAMPLE 2

### Behavioral Profile of P2X₇ Knockout Mice in Models of Depression

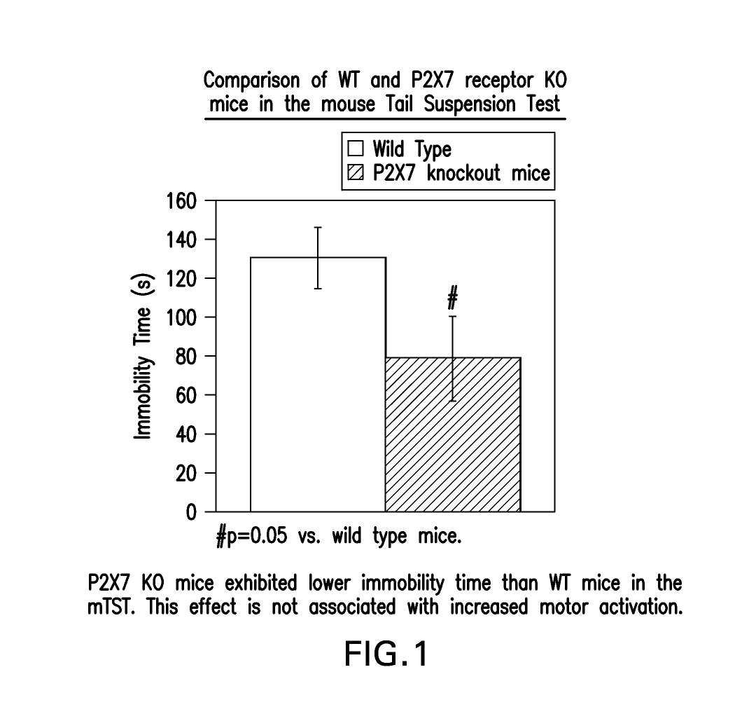

Tail suspension Test (TST): WT mice and P2X₇ knockout mice were acclimated to the testing room for at least 1 hour. A piece of tape was wrapped around the tail, 20 mm from the tip, and the mouse was then hung by the tape from a hook attached to a transducer which communicated information about the duration and animal movement to a computer. The test took 6 minutes. As shown in FIG. 1, P2X₇ knockout mice exhibited lower immobility time than WT mice. The effect was not associated with a nonspecific increase in motor activity.

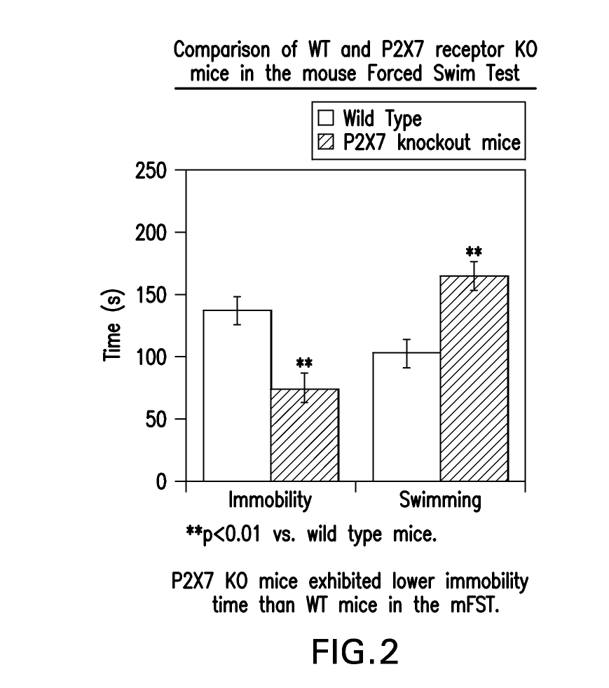

Mouse Forced Swim Test (FST): WT mice and P2X₇ knockout mice were habituated to the testing room for at least 1 h before the experiment. Forced swimming was conducted by individually placing the mice into a container filled with water (23-25° C.) for six minutes. The time spent attempting to escape was recorded. As shown in FIG. 2, P2X7 knockout mice exhibited lower immobility time than WT mice in this test, indicating an antidepressant-like phenotype compared to the WT mice.

What is claimed is:

1. A method of treating a mood disorder comprising administering a therapeutically effective amount of a modulator of P2X₇ receptor activity to a subject suffering from said mood disorder.

2. The method of claim 1 wherein the mood disorder is selected from the group consisting of depressive, bipolar disorder, mood disorder due to a general medical condition, and substance-induced mood disorder.

3. The method of claim 2 wherein the mood disorder is a depressive disorder, wherein the depressive disorder is selected from the group consisting of major depressive disorder and dysthymic disorder.

4. The method of claim 1 wherein the mood disorder is a bipolar disorder, wherein the bipolar disorder is selected from the group consisting of bipolar I disorder, bipolar II disorder and cyclothymic disorder.

5. The method of claim 1 wherein the mood disorder is selected from the group consisting of mood disorder due to a general medical condition, and substance-induced mood disorder.

6. A method of treating an anxiety disorder comprising administering a therapeutically effective amount of a modulator of P2X₇ receptor activity to a subject suffering from said anxiety disorder.

7. The method of claim 6, wherein the anxiety disorders are selected from the group consisting of panic attack, agoraphobia, specific phobia, social phobia, obsessive-compulsive disorder, postraumatic stress disorder, acute stress disorder, and generalized anxiety disorder.

8. The method of claim 1 wherein the modulator of P2X₇ receptor activity is an antagonist.

9. The method of claim 6 wherein the modulator of P2X₇ receptor activity is an antagonist.

10. A pharmaceutical composition comprising a therapeutically effective amount of a modulator of P2X₇ receptor activity or a pharmaceutically acceptable salt or prodrug thereof, and a pharmaceutically acceptable carrier or diluent.

11. The pharmaceutical composition of claim 7, wherein the modulator is an antagonist of the P2X₇ receptor activity.

12. A method of treating a mood disorder comprising administering a therapeutically effective amount of the pharmaceutical composition of claim 11, wherein the mood disorders is selected from the group consisting of depressive, bipolar disorder, mood disorder due to a general medical condition, and substance-induced mood disorder.

13. A method of treating anxiety disorder comprising administering a therapeutically effective amount of the pharmaceutical composition of claim 11, wherein the anxiety disorders is selected from the group consisting of panic attack, agoraphobia, specific phobia, social phobia, obsessive-compulsive disorder, posttraumatic stress disorder, acute stress disorder, and generalized anxiety disorder.

14. A method for treating a patient suffering from, or susceptible to treatment resistant mood disorders comprising administering to said patient an effective amount of a first component which is a P2X7 antagonist or a pharmaceutically acceptable salt thereof, in combination with an effective amount of a second component which is a known antidepressant or a pharmaceutically acceptable salt thereof.

15. A method for treating a patient suffering from anxiety disorders, comprising administering to said patient an effective amount of a first component which is a P2X₇ antagonist or a pharmaceutically acceptable salt thereof, in combination with an effective amount of a second component which is a known antidepressant, tranquilizer, or neuroleptic compound, or a pharmaceutically acceptable salt thereof.
